(19) United States
(12) Patent Application Publication
(10) Pub. No.: US 2026/0363790 A1
(43) Pub. Date: Nov. 19, 2026

(54) NAVIGATION METHOD, DENTAL IMPLANT DEVICE, AND STORAGE MEDIUM

(71) Applicant: Shining 3D Tech Co., Ltd., Hangzhou (CN)

(72) Inventors: Xiaopeng Fu, Hangzhou (CN); Xiaobo Zhao, Hangzhou (CN); Xiaojun Chen, Hangzhou (CN); Huiquan Zhang, Hangzhou (CN)

(21) Appl. No.: 19/721,733

(22) Filed: Jun. 26, 2026

(30) Foreign Application Priority Data
Jun. 30, 2025 (CN) 202510897242.3

(51) Int. Cl. A61B 34/20; A61B 90/00; A61C 8/00

(52) U.S. Cl. CPC A61B 34/20; A61B 90/37; A61C 8/0089; A61B 2034/2055; A61B 2034/2065

(57) ABSTRACT

The present application provides a navigation method, a dental implant device, and a storage medium. The dental implant device includes an image acquisition device and an implant device including an implant head. The implant device is provided with a plurality of first marker features. The navigation method includes acquiring a plurality of first images using an image acquisition device. The plurality of first images are processed to obtain coordinate information of the first marker feature. Pose information of the implant head is determined based on the oral data of the target oral cavity and the coordinate information of the first marker feature. Implant navigation information is displayed in real time based on the pose information of the implant head and the oral data. The method allows clear observation of the internal condition of the oral cavity.

### TECHNICAL FIELD

The present application relates to the technical field of oral implant surgery, and particularly to a navigation method, a dental implant device, and a storage medium.

### BACKGROUND

A dental implant surgery typically uses an extraoral tracking technology to obtain three-dimensional image data of a patient's oral cavity, and the three-dimensional image data is imported into a navigation system to guide an implant operation. However, after obtaining the three-dimensional image data, slight movements of the patient's head may occur, and it may be difficult to observe the internal condition of the oral cavity clearly and in real time using the extraoral tracking technology. The implant position determined by the extraoral tracking technology may be inaccurate, thereby affecting the overall success rate of the dental implant surgery.

### SUMMARY

In one aspect, the present application provides a navigation method applied to a dental implant device, wherein the dental implant device comprises an image acquisition device and an implant device, the implant device comprises an implant head, the implant device is provided with at least one first marker feature, the navigation method comprises: obtaining oral data of a target oral cavity; acquiring, using the image acquisition device attached to the target oral cavity, a plurality of first images; processing the plurality of first images to obtain coordinate information of the first marker feature; determining pose information of the implant head based on the oral data and the coordinate information of the first marker feature; and displaying, in real time, implant navigation information based on the pose information of the implant head and the oral data.

In another aspect, the present application provides a dental implant device, the dental implant device comprises: a storage device storing computer-readable instructions; and a processor configured to execute the computer-readable instructions stored in the storage device to implement the navigation method.

In another aspect, the present application provides a non-transitory storage medium, wherein the non-transitory storage medium stores computer-readable instructions, and the computer-readable instructions are executed by a processor in a dental implant device to implement the navigation method.

### BRIEF DESCRIPTION OF DRAWINGS

FIG. 1 is a partial view of a dental implant device implementing a navigation method according to an embodiment of the present application.

FIG. 2 is a partial view of the dental implant device implementing the navigation method at another position according to an embodiment of the present application.

FIG. 3 is a schematic structural view of the dental implant device implementing the navigation method according to an embodiment of the present application.

FIG. 4 is a flowchart of a navigation method according to an embodiment of the present application.

FIG. 5 is a functional block diagram of a navigation apparatus according to an embodiment of the present application.

The following detailed description will further explain the present application in conjunction with the above drawings.

### DETAILED DESCRIPTION

In order to make the purpose, technical solutions, and advantages of the present application clearer, the present application is described in detail below in conjunction with the drawings and specific embodiments.

It should be noted that, in the present application, “at least one” refers to one or more, and “a plurality of” refers to two or more than two. “And/or” describes an association relationship of associated objects, indicating that three relationships may exist. For example, A and/or B may indicate: A alone exists, A and B exist simultaneously, or B alone exists, where A and B may be singular or plural. The terms “first”, “second”, “third”, “fourth”, etc. (if present) in the specification, claims, and drawings of the present application are used to distinguish similar objects rather than to describe a specific order or sequence.

In the embodiments of the present application, words such as “exemplary” or “for example” are used to indicate an example, illustration, or explanation. Any embodiment or design described as “exemplary” or “for example” in the embodiments of the present application should not be construed as being more preferred or advantageous than other embodiments or designs. Rather, the use of words such as “exemplary” or “for example” is intended to present related concepts in a concrete manner. The following embodiments and features in the embodiments may be combined with each other as long as there is no conflict.

Currently, the dental implant surgery typically uses the extraoral tracking technology to obtain three-dimensional image data of a patient's oral cavity, and the three-dimensional image data is imported into a navigation system to guide an implant operation. However, this method has two problems. In one aspect, after obtaining three-dimensional image data, a patient's head may slightly move, and the extraoral tracking technology makes it difficult to observe the internal condition of the oral cavity in real time and clearly, resulting in inaccurate implant placement and affecting the overall success rate of the dental implant surgery. In another aspect, an extraoral tracking device is bulky and requires a large space, which limits a layout of an operating room, restricts a placement of other medical equipment and an operating space of medical staff, may cause inconvenience during the implant surgery, and reduces a flexibility and an adaptability of the implant surgery.

In order to improve the overall success rate of the surgery and the flexibility and adaptability of the surgery, in some embodiments of the present application, by performing a real-time intraoral scanning to acquire a plurality of first images of the oral cavity to track a first marker feature of an implant head, and determining pose information of the implant head based on oral data and coordinate information of the first marker feature, and then displaying, in real time, implant navigation information based on the pose information of the implant head and the oral data, the problem that the extraoral tracking technology makes it difficult to observe the internal condition of the oral cavity in real time and clearly is avoided, ensuring that the internal condition of the oral cavity can be clearly observed during a implant process, which helps ensure accuracy of an implant position, thereby improving the overall success rate of the surgery.

In addition, because the tracking is performed intraorally, the problem that the extraoral tracking device is bulky and requires a large space, which limits the layout of the operating room, is solved, thereby improving the flexibility and adaptability of the implant surgery.

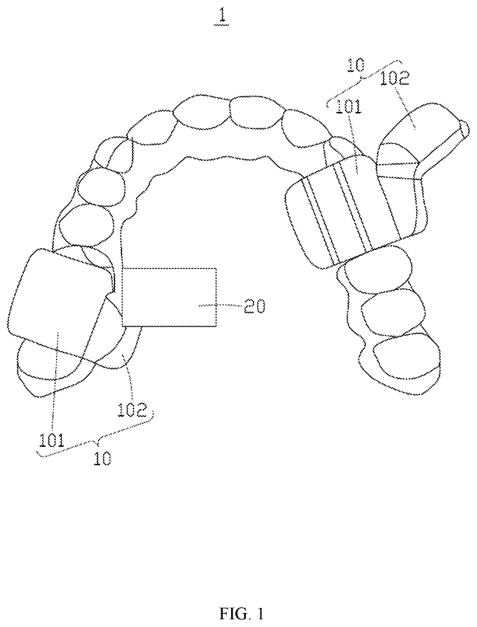

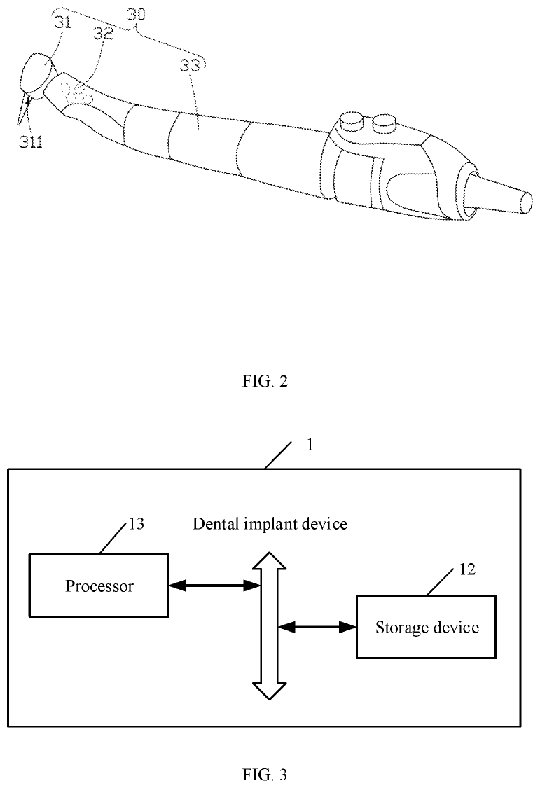

As shown in FIG. 1 and FIG. 2, these are schematic structural views of a dental implant device implementing a navigation method according to an embodiment of the present application.

In some embodiments of the present application, the dental implant device 1 includes a support frame 10, an image acquisition device 20, and an implant device 30. The support frame 10 is mounted inside an oral cavity. The image acquisition device 20 is mounted on the support frame 10. The implant device 30 includes an implant head 31. The implant device 30 is provided with a plurality of first marker features 32. In practical applications, by mounting the image acquisition device 20 on the support frame 10 and mounting the support frame 10 with the image acquisition device 20 inside the oral cavity, and further acquiring images of the oral cavity through the image acquisition device 20, the first marker features 32 are tracked, and pose information of the implant head 31 is determined based on coordinate information of the first marker feature 32 that is tracked, and the implant head 31 is controlled to perform a tooth implantation for a patient based on the pose information, thereby avoiding the problem that the extraoral tracking technology makes it difficult to observe the internal condition of the oral cavity in real time and clearly, ensuring that an internal condition of the oral cavity can be clearly observed during the implant process, which helps ensure accuracy of an implant position, thereby improving the overall success rate of the implant surgery.

In some embodiments of the present application, the support frame 10 is attached to the oral cavity, and the image acquisition device 20 is mounted on the support frame 10. The image acquisition device 20 and the support frame 10 are located inside the oral cavity, or the image acquisition device 20 extends outside the oral cavity through the support frame 10.

In some embodiments of the present application, the support frame 10 can be fixed on a patient's upper jaw, lower jaw, buccal side, or lingual side, etc. Specifically, the support frame 10 can be adjusted according to the position of an implant to be implanted in the patient, where the implant is used to represent a surgical device that replaces a missing tooth.

In some embodiments of the present application, the support frame 10 includes a base 101 and a carrying portion 102. The base 101 is rotatably connected to the carrying portion 102. The carrying portion 102 is configured to carry the image acquisition device 20. A rotatable connection between the base 101 and the carrying portion 102 facilitates rotating the carrying portion 102 and changing an acquisition angle of the image acquisition device 20, ensuring that the internal condition of the oral cavity can be clearly observed during the implant process. For example, the base 101 and the carrying portion 102 can be connected via a ball hinge or a rotating mechanism.

In some embodiments of the present application, the support frame 10 is provided with a second marker feature (not shown in the figures). The second marker feature can be a protrusion, a recess, a protrusion and a recess, a geometric feature, etc. The second marker feature may also be designed without protrusion or recess, which is not limited in this embodiment. For example, the second marker feature can also be an identification line or an identification point. It is understood that the identification line can be a straight line, a wavy line, etc. The second marker feature can be identified by shape, color, coding information, etc., which is not limited in this embodiment. For example, the second marker feature can be a black-and-white identification point. When the second marker feature includes coding information, the coding information can be matched with standard data in a database (such as standard support frame model data, standard identification point data, etc.) to obtain more accurate support frame data.

In some embodiments of the present application, the implant device 30 further includes an implant handpiece 33, and the implant handpiece 33 is connected to the implant head 31. The implant head 31 includes a cutting head 311. For example, when the dental implant device 1 is used to perform a tooth implantation on a patient, the first marker features 32 can be preset on the implant device 30, and the first marker features 32 on the implant handpiece 33 are observed through the image acquisition device 20 to obtain pose information of the cutting head 311. The pose information of the cutting head 311 may include a distance from the cutting head 311 to a target point of the implant and an angle of an axis line of the cutting head 311 relative to a center line of the implant. In some other embodiments, the first marker features 301 can also be provided on the implant head 31, which is not limited in this embodiment. For example, the first marker features 301 can also be provided on the implant handpiece 33.

In some embodiments of the present application, the image acquisition device 20 can be a camera, through which images of the oral cavity can be acquired. The image may include, but is not limited to, information of the first marker features, information of the implant head, implant position information, and information of teeth around the implant.

It is understood that the image acquisition device 20 may not be mounted on the support frame 10, but may be directly mounted inside the oral cavity. Where the image acquisition device 20 is mounted on a dental arch and located inside the oral cavity, or the image acquisition device 20 is mounted on the dental arch and extends outside the oral cavity through its own structure.

In some embodiments of the present application, a three-dimensional model of the patient's oral cavity can be obtained by a scanner. After the three-dimensional model of the patient's oral cavity is obtained, the images acquired by the camera can be aligned with the three-dimensional model of the oral cavity, for example, aligned to a coordinate system where the three-dimensional model of the oral cavity is located, thereby obtaining the pose information of the implant head.

In some embodiments of the present application, a Computed Tomography (CT) device can also be used to obtain CT data including teeth or a Cone Beam Computed Tomography (CBCT) device can also be used to obtain CBCT data including teeth. The images acquired by the camera can be aligned with the CT data or CBCT data, for example, aligned to a coordinate system where the CT data or CBCT data is located, thereby obtaining the pose information of the implant head. Where, the CT data or CBCT data including teeth can also be aligned with the three-dimensional model of the patient's oral cavity obtained by the scanner.

In some embodiments of the present application, the image acquisition device 20 includes a first acquisition component (not shown in the figures) and a second acquisition component (not shown in the figures). The first acquisition component and the second acquisition component are arranged spaced apart from each other. The first acquisition component is configured to capture a tooth region of the patient, to obtain a plurality of third images of the patient's oral cavity, and may be configured to perform a three-dimensional reconstruction, thereby reconstructing tooth three-dimensional data from the acquired images of the tooth region to complete alignment with the oral data (such as three-dimensional model data, CT data, CBCT data, etc.). The second acquisition component is configured to track the implant handpiece 33 to acquire a plurality of fourth images, where the fourth images include the first marker features.

In some embodiments of the present application, by providing two acquisition components spaced apart in the image acquisition device 20, i.e., the first acquisition component and the second acquisition component, images of the oral cavity are acquired from different angles. In one aspect, this facilitates fast and convenient alignment of coordinate system. In another aspect, it facilitates acquiring more accurate images, thereby ensuring the accuracy of the pose information of the implant head.

In some embodiments of the present application, the first acquisition component and the second acquisition component can be the same or different. For example, the first acquisition component can be a black-and-white camera, and the second acquisition component can be a color camera, or the first acquisition component is a color camera and the second acquisition component is a color camera, which is not limited in this embodiment. For example, the first acquisition component and the second acquisition component can also be other devices capable of acquiring images, such as scanning devices, electronic devices (e.g., mobile phones), and other image acquisition devices.

As shown in FIG. 3, this is a schematic structural view of a dental implant device implementing a navigation method according to an embodiment of the present application.

In the embodiment of the present application, the navigation method is applied to one or more dental implant devices 1. The dental implant device 1 is a device capable of automatically performing numerical calculations and/or information processing according to pre-set or stored computer-readable instructions. Its hardware includes, but is not limited to, a microprocessor, an Application Specific Integrated Circuit (ASIC), a Field-Programmable Gate Array (FPGA), a Digital Signal Processor (DSP), an embedded device, etc.

The network to which the dental implant device 1 is connected includes, but is not limited to, the Internet, a wide area network, a metropolitan area network, a local area network, a Virtual Private Network (VPN), etc.

In the embodiment of the present application, the dental implant device 1 further includes, but is not limited to, a storage device 12, a processor 13, and computer-readable instructions stored in the storage device 12 and executable on the processor 13, such as an oral implant surgery program.

It will be understood by those skilled in the art that the schematic diagram is only an example of the dental implant device 1 and does not constitute a limitation on the dental implant device 1. The dental implant device 1 may include more or fewer components than shown, or combine certain components, or different components. For example, the dental implant device 1 may further include an input/output device, a network access device, a bus, etc.

The processor 13 may be a Central Processing Unit (CPU), or other general-purpose processor, a Digital Signal Processor (DSP), an Application Specific Integrated Circuit (ASIC), a Field-Programmable Gate Array (FPGA) or other programmable logic device, a discrete gate or transistor logic device, a discrete hardware component, etc. The general-purpose processor may be any conventional processor such as a microprocessor or a processor, etc. The processor 13 is a computing core and a control center of the dental implant device 1, connecting various parts of the dental implant device 1 using various interfaces and lines, and executing an operating system of the dental implant device 1 and various installed application programs, program codes, etc.

The storage device 12 may be an external storage device and/or an internal storage device of the dental implant device 1. Further, the storage device 12 may be a tangible storage device, such as a memory stick, a Trans-flash Card (TF card), etc.

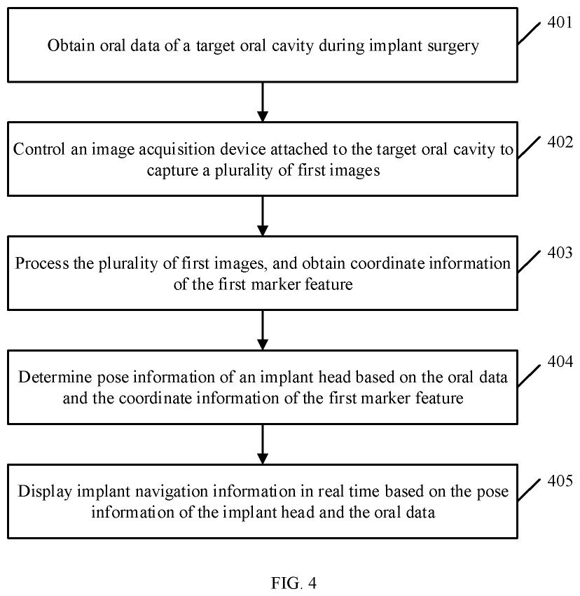

With reference to FIG. 1 to FIG. 3, the storage device 12 in the dental implant device 1 stores computer-readable instructions, and the processor 13 can execute the computer-readable instructions stored in the storage device 12 to implement the navigation method shown in FIG. 4.

As shown in FIG. 4, it is a flowchart of a navigation method according to an embodiment of the present application. The navigation method is applied to a dental implant device (e.g., the dental implant device 1 shown in FIG. 1 to FIG. 3). Depending on different requirements, an order of steps in the flowchart can be changed, and some steps can be omitted. 401, oral data of a target oral cavity is obtained.

In some embodiments of the present application, when performing a tooth implant surgery on a patient, it is necessary to determine information within the patient's oral cavity based on oral data (e.g., three-dimensional model data, CT data, or CBCT data) in order to determine a position of an implant (hereinafter referred to “implant position”).

In some embodiments of the present application, a three-dimensional model of the patient's oral cavity can be obtained by a scanner. Specifically, obtaining the patient's oral data includes: scanning the patient's oral cavity by the scanner in a normal mode to obtain a plurality of first initial images of the oral cavity; scanning the patient's oral cavity by the scanner in a white light mode to obtain a plurality of second initial images of the oral cavity; determining first three-dimensional data based on the plurality of first initial images, and determining second three-dimensional data based on the plurality of second initial images; obtaining three-dimensional reconstruction data based on the first three-dimensional data and the second three-dimensional data; and determining the three-dimensional reconstruction data as the patient's oral data, where the oral data can be high-resolution three-dimensional image data of teeth and surrounding tissue structures of the teeth. The method for obtaining the three-dimensional model of the patient's oral cavity by the scanner is not limited in this embodiment.

In some embodiments of the present application, in the field of tooth implantation, when a scanner is used to acquire images of teeth in the oral cavity for treatment, because the oral cavity includes teeth, and an enamel on a tooth surface has high reflectivity, transmitted light, and diffused light, it may be impossible to acquire an image of the implant position in practical applications, making tooth implantation impossible. In this embodiment, the plurality of first initial images of the oral cavity may be obtained by scanning the oral cavity in the normal mode of the scanner, and the plurality of second initial images of the oral cavity may be obtained by scanning the oral cavity in the white light mode of the scanner.

It is understood that when the oral cavity is scanned in the white light mode of the scanner, the scanner is controlled to project a plurality of white stripe patterns onto the teeth and the implant position in time sequence. Specifically, in the white light mode, the scanner projects the white stripe patterns onto the teeth in time sequence, causing the patterns to be deformed according to a surface shape of the teeth. The deformed patterns are then captured as the plurality of second initial images.

In some embodiments of the present application, after the plurality of first initial images and the plurality of second initial images are obtained, at least one image is obtained from the plurality of first initial images as a first reconstruction image; other images of the plurality of first initial images except the first reconstruction image are used as first encoded images; a three-dimensional reconstruction is performed on the first reconstruction image based on the first encoded images to obtain first three-dimensional data; at least one image is obtained from the plurality of second initial images as a second reconstruction image; other images of the plurality of second initial images except the second reconstruction image are used as second encoded images; a three-dimensional reconstruction is performed on the second reconstruction image based on a sequence of all white stripe patterns in the second encoded images to obtain second three-dimensional data.

In the embodiments of the present application, by merging the first three-dimensional data obtained in the normal mode and the second three-dimensional data obtained in the white light mode, and performing a three-dimensional model reconstruction based on the merged three-dimensional reconstruction data, an intraoral three-dimensional model is obtained, thereby obtaining the oral data. In one aspect, the amount of data for three-dimensional reconstruction of the oral cavity is increased, ensuring the accuracy of the three-dimensional reconstruction data. In another aspect, by using the white light mode to project a plurality of white stripe patterns onto the oral cavity in time sequence, brightness of the acquired stripe images (i.e., the second initial images) is ensured by utilizing the advantage that white light is not easily absorbed by red, yellow, black, and other objects, solving the problem in related technologies that three-dimensional reconstruction data of teeth cannot be obtained due to low brightness of acquired images, improving the accuracy of the obtained three-dimensional reconstruction data of teeth, and thus improving the accuracy of the obtained oral data.

In some other embodiments of the present application, if a scan body is installed in the target oral cavity, the oral data may include data of the scan body. It is understood that the scan body can be installed in a region to be implanted of the target oral cavity, and information of the implant position (hereinafter referred to “implant position information”) of the implant can be accurately determined through the data of the scan body. For example, the scan body can be a characteristic scan body, a coded scan body, etc., which is not limited in this embodiment.

In some embodiments of the present application, after obtaining the oral data of the target oral cavity, the method further includes: determining the implant position information of the implant based on the oral data; determining an installation position of a base based on the implant position information; and adjusting, based on the installation position and the implant position information, an acquisition angle of the image acquisition device by adjusting a carrying portion.

It is understood that the implant position of the implant is obtained from the oral data, the installation position of the base is further determined based on the implant position, and the acquisition angle of the image acquisition device is determined based on the installation position and the implant position information, thereby avoiding the problem that a doctor, after directly viewing the patient's oral cavity, mounts the support frame in the oral cavity based on past experience, which may cause the image acquisition device to fail to capture a desired region, resulting in a low clarity and a completeness of the acquired images of the oral cavity. 402, a plurality of first images are acquired using the image acquisition device.

In some embodiments of the present application, the image acquisition device can be a camera. The camera is controlled to acquire a plurality of first images of the patient's oral cavity in real time, where the first images may include feature information of the implant head.

In some embodiments of the present application, by mounting the image acquisition device inside the patient's oral cavity and acquiring the plurality of first images of the oral cavity in real time, it is ensured that an internal condition of the oral cavity can be clearly observed during a tooth implantation process, thereby improving an overall success rate of the surgery. 403, the plurality of first images are processed to obtain coordinate information of first marker features.

In some embodiments of the present application, after the first images are obtained, in order to improve the efficiency of obtaining coordinate information of the first marker features, processing the plurality of first images to obtain coordinate information of the first marker features includes: determining at least one second image from the plurality of first images, each second image including at least one first marker feature; and processing each second image to obtain the coordinate information of the first marker feature.

In some embodiments of the present application, each second image is preprocessed, the second image is converted into a grayscale image, and a filter is applied to remove noise from the grayscale image to obtain a target image. An edge detection algorithm is then used to identify edges in the target image, and a contour detection algorithm is used to extract a boundary contour of the first marker feature. After the boundary contour is obtained, a two-dimensional coordinate of a center point of the first marker feature is calculated, and the two-dimensional coordinate is determined as the coordinate information of the corresponding first marker feature. 404, pose information of the implant head is determined based on the oral data and the coordinate information of the first marker features.

In some embodiments of the present application, during the tooth implantation process, it is necessary to obtain the pose information of the implant head in real time in order to precisely control the implant head for tooth implantation.

In some embodiments of the present application, determining the pose information of the implant head based on the oral data and the coordinate information of the first marker features includes: converting the coordinate information of the first marker features into a coordinate system where the oral data is located, to obtain first three-dimensional coordinate information of the first marker features; and determining the pose information of the implant head based on the first three-dimensional coordinate information of the first marker features.

In some embodiments of the present application, for each first marker feature, by mapping the coordinate information of the first marker feature into the coordinate system where the oral data is located, the first three-dimensional coordinate information of the first marker feature is obtained.

In some embodiments of the present application, determining the pose information of the implant head based on the first three-dimensional coordinate information of the first marker features includes: obtaining second three-dimensional coordinate information of the implant; determining a position of the implant head and an orientation of the implant head based on the first three-dimensional coordinate information and the second three-dimensional coordinate information; and determining the position (distance information or coordinate information) and the orientation of the implant head as the pose information of the implant head.

In some embodiments of the present application, the position of the implant head is represented by a coordinate of the implant head in a three-dimensional space or represented by a distance to a target position, where the target position may be a target implant position or another preset target position. The orientation of the implant head is represented by angular information of the implant head relative to the implant position.

In some embodiments of the present application, after the first three-dimensional coordinate information and the second three-dimensional coordinate information are obtained, an optimal rotation matrix is solved by singular value decomposition (SVD), and the position and the orientation of the implant head are output according to the solved optimal matrix, thereby obtaining the pose information of the implant head.

In some embodiments of the present application, the first acquisition component performs an image acquisition and obtains tooth data for a three-dimensional reconstruction, the second acquisition component acquires images to obtain the coordinate information of the first marker features. Before the implant surgery, data calibration is required to align the data into a same coordinate system, for example, registering and aligning the image acquisition device to a coordinate system of the oral data. Specifically, the first acquisition component and the second acquisition component are arranged spaced apart from each other, and a pre-position calibration parameter between the first acquisition component and the second acquisition component is obtained. It can be understood that the pre-position calibration parameter refer to a transformation matrix between the first acquisition component and the second acquisition component, which describes a relative spatial relationship between the first acquisition component and the second acquisition component. Determining pose information of the implant head based on the oral data and the coordinate information of the first marker features includes: obtaining a plurality of third images acquired by the first acquisition component; processing each of the plurality of third images to obtain tooth data of at least a portion of teeth; aligning the tooth data of the at least a portion of teeth with the oral data, and unifying the oral data and the coordinate information of the first marker features into a same coordinate system based on the pre-position calibration parameter, to determine the pose information of the implant head. For example, the tooth data of the at least a portion of teeth is aligned to the coordinate system of the oral data through a first transformation relationship. The first transformation relationship refers to a transformation matrix between a coordinate system of the tooth data acquired by the first acquisition component and the coordinate system of the oral data. Based on the pre-position calibration parameter between the two acquisition components and the first transformation relationship, the coordinate information of the first marker features acquired by the second acquisition component can be transformed into the coordinate system of the oral data. Since a positional relationship between the two acquisition components is fixed, data relationship (e.g., a transformation relationship between coordinate systems) between the two acquisition components can be determined through a position calibration. The position calibration can be performed by scanning or photographing a calibration device. The method of position calibration is not limited herein. Then, based on the position calibration parameter and the first transformation relationship, the relationship between each acquisition component and the coordinate system of the oral data can be known. On this basis, by processing the fourth images acquired by the second acquisition component to obtain the coordinate information of the first marker features, the coordinate information of the first marker features can be transformed into the coordinate system of the oral data.

During the implantation process, the second acquisition component acquires first images, and real-time coordinate information of the first marker features is obtained based on the first images or the second images; the coordinate information is converted into the coordinate system of the oral data to obtain the first three-dimensional coordinate information of the first marker features; the second three-dimensional coordinate information of the implant is obtained; a position and an orientation of the implant head are determined based on the first three-dimensional coordinate information and the second three-dimensional coordinate information; the position and the orientation of the implant head are determined as the pose information of the implant head.

In some embodiments of the present application, by providing two acquisition components spaced apart in the image acquisition device 20, i.e., the first acquisition component and the second acquisition component, images of the oral cavity are acquired from different angles. In one aspect, this facilitates fast and convenient coordinate system alignment. In another aspect, it facilitates acquiring more accurate images, thereby ensuring the accuracy of the obtained pose information of the implant head.

In some embodiments of the present application, calibration and alignment can also be performed as needed during the implant surgery to further ensure the accuracy of the pose information of the implant head.

In some embodiments of the present application, the processing of the fourth images is similar to the processing of the first images. The fourth image is essentially the first image.

In some other embodiments of the present application, data calibration can be performed as needed before or during the implant surgery using tooth data of several teeth and part data of the support frame to align the data into the same coordinate system. Specifically, the support frame is provided with a plurality of second marker features, and the second marker features are pre-calibrated with a scanner (e.g., an intraoral scanner). Determining pose information of the implant head based on the oral data and the coordinate information of the first marker features includes: obtaining tooth data of several teeth and coordinate data of the second marker features; where the tooth data of the several teeth and the coordinate data of the second marker features can be obtained by the scanner. By aligning the tooth data of the several teeth with the oral data, the oral data and the coordinate information of the first marker features are unified into the same coordinate system. For example, since the tooth data of the several teeth and the coordinate data of the second marker features are in the same coordinate system, the tooth data of the several teeth is aligned to the coordinate system of the oral data through a second transformation relationship. Based on the second transformation relationship, the coordinate data of the second marker features can be transformed into the coordinate system of the oral data. On this basis, the coordinate system of the image acquisition device fixed to the support frame (e.g., a camera coordinate system) can be registered and aligned with the coordinate system of the oral data, and then the coordinate information of the first marker features acquired by the image acquisition device can be transformed into the coordinate system of the oral data.

During the implantation process, the image acquisition device acquires first images, and real-time coordinate information of the first marker features is obtained based on the first images or the second images; the coordinate information is converted into the coordinate system of the oral data to obtain the first three-dimensional coordinate information of the first marker features; the second three-dimensional coordinate information of the implant is obtained; the position and orientation of the implant head are determined based on the first three-dimensional coordinate information and the second three-dimensional coordinate information; the position and orientation of the implant head are determined as the pose information of the implant head.

The above two examples of methods for registering and aligning coordinate systems are merely illustrative, and practical applications are not limited thereto.

405, implant navigation information is displayed in real time based on the pose information of the implant head and the oral data.

In some embodiments of the present application, after the pose information of the implant head and the oral data are obtained, the implant navigation information can be displayed in real time. For example, the implant navigation information can be displayed on a display screen of the dental implant device, or on an electronic device. The implant navigation information may be a navigation image or a navigation video, and may also include voice information. The implant navigation information includes real-time oral data and a pose of the implant head. Further, a navigation planning path for the implant can be displayed by an image or a video, or navigation information such as a prompt for a distance and angle for moving from a current position of the implant handpiece to a target tooth position can be provided.

In some embodiments of the present application, by mounting the image acquisition device inside the patient's oral cavity, performing real-time intraoral scanning to acquire a plurality of first images of the oral cavity to track the first marker features of the implant head, determining the pose information of the implant head based on the oral data and the coordinate information of the first marker features, and then displaying, in real time, implant navigation information based on the pose information of the implant head and the oral data, the problem that the extraoral tracking technique makes it difficult to observe the internal condition of the oral cavity in real time and clearly is avoided. By installing the image acquisition device intraorally, the intraoral condition can be observed more intuitively without being obstructed, ensuring that the internal condition of the oral cavity can be clearly observed during the implant process, which helps ensure accuracy of the implant position, thereby improving the overall success rate of the surgery.

In addition, because the tracking is performed intraorally, the problem that the extraoral tracking device is bulky and requires a large space, which limits the layout of the operating room, is solved, and the device is easy to carry and can be used between clinics or dental chairs, thereby improving the flexibility and adaptability of the implant surgery.

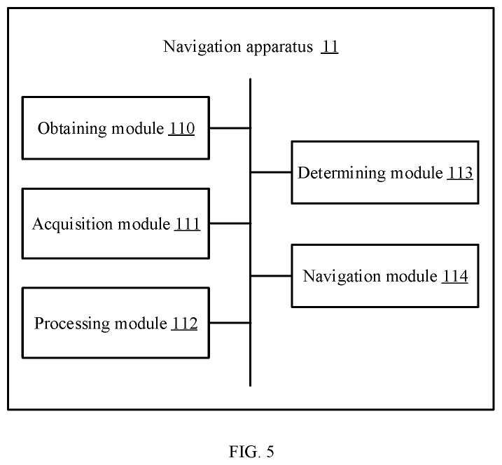

As shown in FIG. 5, this is a functional block diagram of a navigation apparatus according to an embodiment of the present application. The navigation apparatus 11 is operable on a dental implant device. The dental implant device includes a storage device, and the storage device includes a plurality of storage units. The navigation apparatus 11 includes an obtaining module 110, an acquisition module 111, a processing module 112, a determining module 113, and a navigation module 114. The modules/units referred to in the present application refer to a series of computer-readable instruction segments that can be executed by the processor 13 and are capable of performing fixed functions, and the instruction segments are stored in the storage device 12.

The obtaining module 110 is configured to obtain oral data of a target oral cavity.

The acquisition module 111 is configured to obtain a plurality of first images of the target oral cavity acquired by the image acquisition device.

The processing module 112 is configured to process the first images to obtain coordinate information of the first marker feature.

The determining module 113 is configured to determine pose information of the implant head based on the oral data and the coordinate information of the first marker feature.

The navigation module 114 is configured to display, in real time, implant navigation information based on the pose information of the implant head and the oral data.

If the integrated modules/units of the dental implant device 1 are implemented in the form of software functional units and sold or used as independent products, they may be stored in a computer-readable storage medium. Based on such understanding, all or part of the processes in the methods of the above embodiments of the present application may be implemented by computer-readable instructions instructing relevant hardware. The computer-readable instructions may be stored in a computer-readable storage medium. When the computer-readable instructions are executed by a processor, the steps of the above method embodiments may be implemented.

Where the computer-readable instructions include computer-readable instruction code. The computer-readable instruction code may be in source code form, object code form, an executable file, or some intermediate form. The computer-readable medium may include: any entity or device capable of carrying the computer-readable instruction code, a recording medium, a USB flash drive, a removable hard disk, a magnetic disk, an optical disk, a computer memory, a Read-Only Memory (ROM), a Random Access Memory (RAM).

The storage device 12 may be used to store computer-readable instructions and/or modules. The processor 13 implements various functions of the dental implant device 1 by running or executing the computer-readable instructions and/or modules stored in the storage device 12 and calling data stored in the storage device 12. The storage device 12 may mainly include a program storage area and a data storage area. The program storage area may store an operating system, an application required for at least one function (such as a sound playback function, an image playback function, etc.). The data storage area may store data created according to the use of the dental implant device, etc. The storage device 12 may include non-volatile and volatile storage devices, for example: a hard disk, memory, a plug-in hard disk, a Smart Media Card (SMC), a Secure Digital (SD) card, a Flash Card, at least one magnetic disk storage device, a flash memory device, or other storage devices.

Exemplarily, the computer-readable instructions may be divided into one or more modules/units. The one or more modules/units are stored in the storage device 12 and executed by the processor 13 to complete the present application. The one or more modules/units may be a series of computer-readable instruction segments capable of performing specific functions, and the instruction segments are used to describe the execution process of the computer-readable instructions in the dental implant device 1. For example, the computer-readable instructions may be divided into the obtaining module 110, the acquisition module 111, the processing module 112, the determining module 113, and the navigation module 114.

For detailed content of the functions of each module/unit, reference may be made to the detailed description of FIG. 3 to FIG. 5 above, which will not be repeated here.

In some embodiments of the present application, by mounting the image acquisition device inside the patient's oral cavity, performing real-time intraoral scanning to acquire a plurality of first images of the oral cavity to track the first marker features of the implant head, determining the pose information of the implant head based on the oral data and the coordinate information of the first marker features, and then displaying, in real time, implant navigation information based on the pose information of the implant head and the oral data, the problem that the extraoral tracking technique makes it difficult to observe the internal condition of the oral cavity in real time and clearly is avoided, ensuring that the internal condition of the oral cavity can be clearly observed during the implant process, which helps ensure accuracy of the implant position, thereby improving the overall success rate of the surgery.

In addition, because the tracking is performed intraorally, the problem that the extraoral tracking device is bulky and requires a large space, which limits the layout of the operating room, is solved, thereby improving the flexibility and adaptability of the implant surgery.

In the several embodiments provided in the present application, it should be understood that the disclosed system, apparatus, and method may be implemented in other ways. For example, the apparatus embodiments described above are merely illustrative. For instance, the division of modules is only a logical function division, and there may be other division manners in actual implementation.

Modules described as separate components may or may not be physically separate. Components shown as modules may or may not be physical units, i.e., they may be located in one place or distributed over multiple network units. Some or all of the modules may be selected according to actual needs to achieve the purpose of the solution of the embodiment.

In addition, functional modules in the various embodiments of the present application may be integrated into one processing unit, or each module may exist separately physically, or two or more modules may be integrated into one unit. The integrated unit may be implemented in the form of hardware or in the form of hardware plus software functional modules.

Therefore, from any point of view, the embodiments should be regarded as exemplary and non-limiting. The scope of the present application is defined by the appended claims rather than the above description. All changes falling within the meaning and range of equivalents of the claims are intended to be included in the present application. Any reference numerals in the claims should not be construed as limiting the claims involved.

In addition, it is obvious that the word “comprising” does not exclude other units or steps, and the singular does not exclude a plurality. A plurality of units or devices may also be implemented by one unit or device via software or hardware. The terms “first”, “second”, etc. are used to denote names and do not indicate any specific order.

What is claimed is:

1. A navigation method applied to a dental implant device, which comprises an image acquisition device and an implant device, the implant device comprising an implant head, the implant device being provided with at least one first marker feature, the navigation method comprising:
obtaining oral data of a target oral cavity;
controlling the image acquisition device attached to the target oral cavity to capture a plurality of first images;
processing the plurality of first images, and obtaining coordinate information of the first marker feature;
determining pose information of the implant head based on the oral data and the coordinate information of the first marker feature; and
displaying implant navigation information in real time based on the pose information of the implant head and the oral data.

2. The navigation method of claim 1, wherein processing the plurality of first images and obtaining coordinate information of the first marker feature comprises:
determining at least one second image from the plurality of first images, each of the at least one second image comprising the first marker feature;
processing each of the at least one second image and obtaining the coordinate information of the first marker feature.

3. The navigation method of claim 1, wherein determining pose information of the implant head based on the oral data and the coordinate information of the first marker feature comprises:
converting the coordinate information of the first marker feature into a coordinate system of the oral data, and obtaining first three-dimensional coordinate information of the first marker feature;
determining the pose information of the implant head based on the first three-dimensional coordinate information of the first marker feature.

4. The navigation method of claim 3, wherein determining the pose information of the implant head based on the first three-dimensional coordinate information of the first marker feature comprises:
obtaining second three-dimensional coordinate information of an implant inside the target oral cavity;
determining a position and an orientation of the implant head based on the first three-dimensional coordinate information and the second three-dimensional coordinate information;
determining the position and the orientation of the implant head as the pose information of the implant head.

5. The navigation method of claim 1, wherein the image acquisition device comprises a first acquisition component and a second acquisition component, the first acquisition component and the second acquisition component are arranged spaced apart from each other, the first acquisition component is configured to obtain tooth data of the target oral cavity, and the second acquisition component is configured to obtain the coordinate information of the first marker feature;
wherein determining pose information of the implant head based on the oral data and the coordinate information of the first marker feature comprises:
acquiring a plurality of third images using the first acquisition component;
processing the plurality of third images, and obtaining tooth data of at least a portion of teeth;
determining the pose information of the implant head by aligning the tooth data of the at least a portion of teeth with the oral data, and unifying the oral data and the coordinate information of the first marker feature into a same coordinate system based on a pre-position calibration parameter between the first acquisition component and the second acquisition component.

6. The navigation method of claim 1, wherein the dental implant device further comprises a scanner and a support frame, the support frame is provided with a plurality of second marker features, the image acquisition device is mounted on the support frame; wherein determining the pose information of the implant head based on the oral data and the coordinate information of the first marker feature comprises:
obtaining, by the scanner, tooth data of at least a portion of teeth of the target oral cavity and coordinate data of the second marker feature;
determining the pose information of the implant head by aligning the tooth data of the at least a portion of teeth with the oral data, and unifying the oral data and the coordinate information of the first marker feature into a same coordinate system based on the coordinate data of the second marker feature.

7. The navigation method of claim 1, wherein the dental implant device further comprises a support frame, the support frame is attached to the target oral cavity, the image acquisition device is attached on the support frame;
wherein the image acquisition device and the support frame are located inside the target oral cavity, or the image acquisition device extends outside the target oral cavity through the support frame.

8. The navigation method of claim 6, wherein the support frame comprises a base and a carrying portion, the base is rotatably connected to the carrying portion, the carrying portion is configured to carry the image acquisition device; wherein after obtaining the oral data of the target oral cavity, the method further comprises:
obtaining implant position information of an implant inside the target oral cavity based on the oral data;
determining an installation position of the base based on the implant position information;
based on the installation position and the implant position information, adjusting an acquisition angle of the image acquisition device by adjusting the carrying portion.

9. The navigation method of claim 8, wherein the implant placement information is determined based on scan body data of the oral data.

10. The navigation method of claim 1, wherein obtaining the oral data of the target oral cavity comprises:
scanning the target oral cavity by a scanner in a normal mode, and obtaining a plurality of first initial images;
scanning the target oral cavity by the scanner in a white light mode, and obtaining a plurality of second initial images;
determining first three-dimensional data based on the plurality of first initial images;
determining second three-dimensional data based on the plurality of second initial images;
obtaining three-dimensional reconstruction data based on the first three-dimensional data and the second three-dimensional data; and
determining the three-dimensional reconstruction data as the oral data.

11. The navigation method of claim 1, wherein the implant device further comprises an implant handpiece connected to the implant head, and the at least one first marker feature is provided on the implant handpiece or on the implant head.

12. The navigation method of claim 1, wherein displaying the implant navigation information in real time comprises:
displaying the implant navigation information on a display screen of the dental implant device or on an electronic device, the implant navigation information comprising at least one of a navigation image, a navigation video, and voice information.

13. A dental implant device, comprising:
an image acquisition device;
an implant device comprising an implant head and being provided with at least one first marker feature;
at least one processor; and
a storage device being stored with computer-readable instructions, which when executed by the at least one processor, causes the at least one processor to:
obtain oral data of a target oral cavity;
control the image acquisition device attached to the target oral cavity to capture a plurality of first images;
process the plurality of first images, and obtain coordinate information of the first marker feature;
determine pose information of the implant head based on the oral data and the coordinate information of the first marker feature; and
display implant navigation information in real time based on the pose information of the implant head and the oral data.

14. The dental implant device of claim 13, wherein the at least one processor processes the plurality of first images and obtains coordinate information of the first marker feature by:
determining at least one second image from the plurality of first images, each of the at least one second image comprising the first marker feature;
processing each of the at least one second image and obtaining the coordinate information of the first marker feature.

15. The navigation method of claim 13, wherein the at least one processor determines pose information of the implant head based on the oral data and the coordinate information of the first marker feature by:
converting the coordinate information of the first marker feature into a coordinate system of the oral data, and obtaining first three-dimensional coordinate information of the first marker feature;
determining the pose information of the implant head based on the first three-dimensional coordinate information of the first marker feature.

16. The navigation method of claim 15, wherein the at least one processor determines the pose information of the implant head based on the first three-dimensional coordinate information of the first marker feature by:
obtaining second three-dimensional coordinate information of an implant inside the target oral cavity;
determining a position and an orientation of the implant head based on the first three-dimensional coordinate information and the second three-dimensional coordinate information;
determining the position and the orientation of the implant head as the pose information of the implant head.

17. A non-transitory storage medium, having computer-readable instructions stored thereon, wherein when the computer-readable instructions are executed by a processor of a dental implant device, a navigation method is implemented; wherein the dental implant device comprises an image acquisition device and an implant device comprising an implant head; wherein the implant device is provided with at least one first marker feature, and the navigation method comprises:
obtaining oral data of a target oral cavity;
controlling the image acquisition device attached to the target oral cavity to capture a plurality of first images;
processing the plurality of first images, and obtaining coordinate information of the first marker feature;
determining pose information of the implant head based on the oral data and the coordinate information of the first marker feature; and
displaying implant navigation information in real time based on the pose information of the implant head and the oral data.

18. The non-transitory storage medium of claim 17, wherein processing the plurality of first images and obtaining coordinate information of the first marker feature comprises:
determining at least one second image from the plurality of first images, each of the at least one second image comprising the first marker feature;
processing each of the at least one second image and obtaining the coordinate information of the first marker feature.

19. The non-transitory storage medium of claim 17, wherein determining pose information of the implant head based on the oral data and the coordinate information of the first marker feature comprises:
converting the coordinate information of the first marker feature into a coordinate system of the oral data, and obtaining first three-dimensional coordinate information of the first marker feature;
determining the pose information of the implant head based on the first three-dimensional coordinate information of the first marker feature.

20. The non-transitory storage medium of claim 19, wherein determining the pose information of the implant head based on the first three-dimensional coordinate information of the first marker feature comprises:
obtaining second three-dimensional coordinate information of an implant inside the target oral cavity;
determining a position and an orientation of the implant head based on the first three-dimensional coordinate information and the second three-dimensional coordinate information;
determining the position and the orientation of the implant head as the pose information of the implant head.
